(19) United States
(12) Patent Application Publication
(10) Pub. No.: US 2014/0066802 A1
(43) Pub. Date: Mar. 6, 2014

(54) Cognition and Usability Aptitude Evaluations for Clinician Programmers

(71) Applicants: Norbert Kaula, Arvada, CO (US); Yohannes Iyassu, Denver, CO (US); Paul Landers, Broomfield, CO (US); Jeff Peters, Denver, CO (US); Carl Mosley, Ormond Beach, FL (US)

(72) Inventors: Norbert Kaula, Arvada, CO (US); Yohannes Iyassu, Denver, CO (US); Paul Landers, Broomfield, CO (US); Jeff Peters, Denver, CO (US); Carl Mosley, Ormond Beach, FL (US)

(73) Assignee: Greatbatch Ltd., Clarence, NY (US)

(21) Appl. No.: 13/600,943

(22) Filed: Aug. 31, 2012

(51) Int. Cl. A61B 5/00; A61B 5/05; A61B 5/11

(52) U.S. Cl. 600/554; 600/558; 600/557

(57) ABSTRACT

The present disclosure involves an electronic device. The electronic device is configured to perform evaluations on a patient user for medical purposes. The electronic device includes a touchscreen display configured to receive input from the user. The electronic device includes a memory storage component configured to store programming code. The electronic device includes a computer processor configured to execute the programming code to perform an evaluation of the user's mental and physical abilities. The evaluation includes prompting the user to perform a plurality of tasks. At least one of the tasks prompts the user to manipulate one or more graphical models shown on the touchscreen display according to predefined instructions. The evaluation includes detecting, via the touchscreen display, responses from the user for the tasks. The evaluation includes determining, based on the detected responses, whether the user is mentally and physically fit to provide reliable feedback to medical personnel.

### BACKGROUND

As medical device technologies continue to evolve, active implanted medical devices have gained increasing popularity in the medical field. For example, one type of implanted medical device includes neurostimulator devices, which are battery-powered or battery-less devices that are designed to deliver electrical stimulation to a patient. Through proper electrical stimulation, the neurostimulator devices can provide pain relief for patients or restore bodily functions.

There are situations where it is desirable and necessary for healthcare professionals to obtain feedback from a patient to successfully determine a diagnosis or provide a therapy. However, accurate and meaningful feedback requires the patient to have sufficient mental and physical abilities. Therefore, healthcare professionals may implement a series of tests to evaluate the patient's mental and physical abilities. Existing methods and mechanisms for conducting such evaluations may suffer from various drawbacks. The standardized tests are not sufficiently integrated or geared towards medical devices or pain treatment. Also, these tests are not sufficiently embedded into medical devices that provide that type of treatment and check whether the patient is able to use the device, touch screen usage, or other electronic means. And the test, because it is embedded into the device, may be used in an intra-op, pre-op, or post-op, and may be boring to the patient.

Therefore, although existing methods and mechanisms for evaluating a patient's mental and physical abilities have been generally adequate for their intended purposes, they have not been entirely satisfactory in every aspect.

### SUMMARY

One aspect of the present disclosure involves an electronic device. The electronic device includes: a touchscreen display configured to receive input from a user; a memory storage component configured to store programming code; and a computer processor configured to execute the programming code to perform an evaluation of the user's mental and physical abilities, wherein the evaluation includes: prompting the user to perform a plurality of tasks, wherein at least one of the tasks prompts the user to manipulate one or more graphical models shown on the touchscreen display according to predefined instructions; detecting, via the touchscreen display, responses from the user for the tasks; and determining, based on the detected responses, whether the user is mentally and physically fit to provide reliable feedback to medical personnel.

Another aspect of the present disclosure involves a medical system. The medical system includes: a first communications module configured to instruct a patient to perform a series of tasks designed to evaluate the patient's cognitive and tactile functions; an input detection module configured to detect the patient's performance of the tasks, including detecting clicking and dragging movements made by the patient on a touchscreen; a processing and analysis module configured to analyze the patient's performance of the tasks and in response thereto produce an evaluation of the patient's cognitive and tactile functions; and a second communications module configured to display a representation of the evaluation of the patient's cognitive and tactile functions to a medical professional.

Yet another aspect of the present disclosure involves method of interacting with a user. The method includes: communicating instructions to the user via an electronic device having a touch-sensitive user interface, the instructions requesting the user to manipulate one or more visual objects through the user interface by generating the one or more visual objects, moving the one or more visual objects, or altering an attribute of the one or more visual objects; detecting, via the user interface, input from the user in response to the instructions; and determining whether the user is able to provide dependable medical feedback.

One more aspect of the present disclosure involve a method of evaluating a patient's mental and physical abilities. The method includes: administering, at least in part using an electronic device that contains a touchscreen interface, a series of tests to the patient requesting the patient to perform at least one of the following actions via the touchscreen interface: producing an image having a specified geometry; moving an image from a first location to a specified second location; resizing or reshaping an image; shading an image with a specified color; and clicking on a set of images according to a specified sequence. These actions are performed to see if the patient can recognize the question and translate the question into a drawing on a touchscreen device. These actions are also performed to see if the patient can operate a touchscreen, to manipulate objects using the touchscreen, and to see whether the patient can execute a program (e.g., a program within a pulse generator), or if the patient is capable of portraying pain/stimulation perception into a computerized representation using the touchscreen. The method also includes receiving an evaluation of the patient's mental and physical capabilities based on the patient's response to the series of tests; and determining whether the patient is capable of drawing a pain map or a stimulation map representing pain or stimulation experienced by the patient.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In the figures, elements having the same designation have the same or similar functions.

FIG. 1 is a simplified block diagram of an example medical environment in which evaluations of a patient may be conducted according to various aspects of the present disclosure.

FIG. 2 is a flowchart illustrating a process flow for implementing an evaluation of a patient according to various aspects of the present disclosure.

FIG. 3 is a flowchart illustrating a method for performing a Cognition Evaluation according to various aspects of the present disclosure.

FIGS. 4-11 are example screenshots of a user interface for performing the Cognition Evaluation according to various aspects of the present disclosure.

FIG. 12 is a flowchart illustrating a method for performing a Usability Aptitude Evaluation according to various aspects of the present disclosure.

FIGS. 13-15 are example screenshots of a user interface for performing the Usability Aptitude Evaluation according to various aspects of the present disclosure.

FIG. 16 is a flowchart illustrating a method of interacting with a user to determine the quality of the user's feedback.

FIG. 17 is a flowchart illustrating a method of evaluating a patient's mental and physical abilities.

FIG. 18 is a simplified block diagram of a medical system according to various aspects of the present disclosure.

FIG. 19 is a simplified block diagram of an electronic programmer according to various aspects of the present disclosure.

FIGS. 20A and 20B are side and posterior views of a human spine, respectively.

### DETAILED DESCRIPTION

It is to be understood that the following disclosure provides many different embodiments, or examples, for implementing different features of the invention. Specific examples of components and arrangements are described below to simplify the present disclosure. These are, of course, merely examples and are not intended to be limiting. Various features may be arbitrarily drawn in different scales for simplicity and clarity.

In recent years, the use of active implanted medical devices has become increasingly prevalent. Some of these implanted medical devices include neurostimulator devices that are capable of providing pain relief by delivering electrical stimulation to a patient. The patient may use an electronic programmer (such as a patient programmer) to configure the neurostimulator device, so that a desired stimulation therapy can be delivered to the patient.

To ensure that the patient is capable of using the programmer to perform certain tasks, such as providing accurate feedback to a surgeon, healthcare professionals may evaluate the patient's mental and physical state through a series of tests. These tests allow the healthcare professionals to assess the patient's cognitive and motor functions. A patient should do well on the tests before he is deemed capable of using the programmer, because a patient with insufficient cognitive abilities and/or motor skills may not be able to provide relevant or reliable feedback to medical personnel. For example, such patient may not be able to generate an accurate pain or stimulation map representing the pain or stimulation experienced by the patient.

Current methods and mechanisms for conducting patient evaluations may suffer from various shortcomings. These shortcomings include:
The tests may not be sufficiently standardized for the specific environment discussed herein. In other words, the tests may vary from provider to provider and environment to environment.
The tests are not geared or exist on the programming devices (such as clinician/patient programmers)
The test may not give a full representation of the cognitive state of the patient.
The tests that utilize touchscreen electronic devices may still rely too much on verbal communication and may not be “hands on” enough.
The tests may not engage the attention of the patient and may not be applicable enough to the desired type of therapy.
The patient's introduction to the tests may be inadequate or incomplete.
   Due to these shortcomings, the current methods and mechanisms for evaluating the patient's mental state and physical state are not satisfactory in many respects.

According to the various aspects of the present disclosure, a two-pronged patient evaluation method is disclosed. One prong of the method involves evaluating the mental functions of a user, for example a user who is a patient (and thereafter referred to as such). Tests may be done to evaluate whether the patient generally has the capability of providing accurate and meaningful feedback (such as recording a pain or stimulation map or responding to questions accurately), and to evaluate specifically whether the patient is sufficiently lucid during or after a medical procedure (e.g., a surgery) to provide such feedback. This prong of the method may be referred to as a “Cognition Evaluation.”

The other prong of the method generally pertains to a patient's ability to use a suitable mechanism to give input or feedback to medical personnel. In particular, tests may be done to assess the patient's ability to use a device through which the patient will provide input or feedback. For example, a patient may be expected to eventually provide feedback such as the location and shape of a pain map or a stimulation map to a surgeon. The patient may be expected to use an electronic programmer such as a clinician programmer or a patient programmer to provide such feedback. In other words, the patient is programming himself/herself. In that case, the patient's ability to use these programmers is tested by this prong. The patient may be prompted to manipulate models in ways that simulate the tasks the patient is expected to perform when he is eventually expected to give feedback. This prong of the method may be referred to as a “Usability Aptitude Evaluation.”

According to various aspects of the present disclosure, both the Cognition Evaluation prong and the Usability Aptitude Evaluation prong can be implemented in a portable handheld electronic device. In other words, the patient will undergo these evaluations by manipulating the electronic device according to a set of instructions. In some embodiments, the electronic device may be an electronic programmer (such as a clinician programmer or a patient programmer) used to configure the implanted medical device. In other embodiments, the electronic device may include a simulator that can simulate the interface of a clinician programmer or a patient programmer to teach or demonstrate the functionality of a programmer to a patient. For example, the electronic device may include a tablet having an electronic user interface that simulates the interface of the clinician or patient programmer. The cognition evaluation and the usability aptitude evaluation will now each be discussed in more detail.

### I. Cognition Evaluation

As a part of the Cognition Evaluation, the patient may be presented a standard set of cues and tasks to test the patient's cognitive abilities both verbally and non-verbally. Patient input for the evaluation may include, but is not limited to, tactile (touch screen) input or aural (spoken) input, or external devices such as a patient feedback device. Other methods for transmission and reception of patient input or feedback may include, but are not limited to, eye and eyelid movement, jaw or lingual movement, response to light-emitting diodes (LEDs), video or motion capture, and nascent technologies.

Attending healthcare professionals may be able to view patient input, as well as stimulation parameters or other information, on corresponding monitors. In some embodiments, to capture the patient's attention more fully, the evaluation may be designed to be game-like in form.

A cumulative score with a maximum of X points may be calculated based on patient responses, where X is a function of a series of sub-scores that each measure the result of the patient response to an individual task or test. The sub-scores may or may not be weighted. In some embodiments, scores are viewable by the healthcare professionals but are not viewable by the patient. A cumulative score of Y (where Y<X) or less may indicate cognitive impairment. In certain embodiments, no judgment about patient abilities will be presented to the healthcare professional: only the score shall be presented. The healthcare professional may then determine the patient's mental and physical abilities based on the score received. This approach promotes flexible, yet informed, decisions by healthcare professionals.

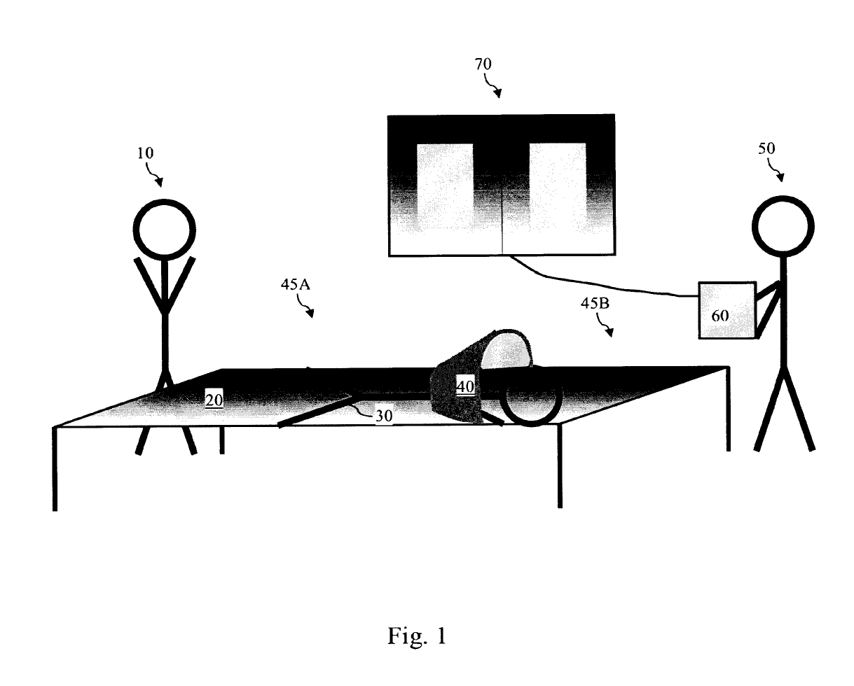

The above elements of the Cognition Evaluation are illustrated graphically in FIGS. 1-11, which are discussed in more detail below.

FIG. 1 is a simplified diagram illustrating an example environment in which an embodiment of the Cognitive Evaluation may be performed. In FIG. 1, the example environment includes an operating room where a surgical procedure is performed by a surgeon 10. An operating table 20 may be provided. A patient 30 for which the surgical procedure is performed is lying on the operating table 20. A sterile shield 40 surrounds the patient 30. The sterile shield 40 may help divide the operating room into a sterile portion 45A and a non-sterile portion 45B. The surgeon 10 is located in the sterile portion 45A of the operating room. A healthcare professional 50 is located in the non-sterile portion 45B of the operating room.

At any time before, during, or after the surgery, the healthcare professional 50 may evaluate the mental state of the patient 30 through an electronic device 60, which is a portable and handheld device in the illustrated embodiment. As discussed above, the electronic device 60 is capable of simulating a user interface through which the patient is expected to eventually provide input or feedback to the surgeon 10 and/or the healthcare professional 50. In some embodiments, the electronic device 60 includes a clinician programmer. In other embodiments, the electronic device 60 may include a patient programmer or a suitable electronic tablet. The patient 30 may undergo the evaluation by performing appropriate manipulations on the electronic device 60. In other alternative embodiments, a device outside of (or external to) the electronic device 60 may be used to perform the cognitive testing as well. For example, stationary or screens embedded into the operating table may be used as such device.

Meanwhile, a monitor 70 may be used to display patient input on the electronic device 60 as well as stimulation parameters or other relevant information. The monitor 70 may include a dual screen (or split screen) display in some embodiments. The monitor 70 is viewable by the surgeon 10 and the healthcare professional 50 but may not be viewable by the patient 30. The monitor 70 may be communicatively coupled to the electronic device 60, for example through a suitable wired or wireless telecommunications protocol. In this manner, the surgeon 10 (or the healthcare professional 50) may be able to view the patient's feedback (for example a drawing of a stimulation map or a pain map) without requiring direct access to the electronic device 60. Additional aspects of the monitor 70 may be found in U.S. patent application Ser. No. 13/638,142, entitled “Clinician Programming System and Method,” and filed on Aug. 31, 2012, the entirety of which is herein incorporated by reference.

Although FIG. 1 illustrates a single electronic device 60 that is “shared” by the patient 30 and the healthcare professional 50, it is understood that multiple electronic devices similar to the electronic device 60 may be used to conduct the patient evaluations in alternative embodiments. For example, the patient 30 may have access to a first programmer (such as a clinician or patient programmer), and the healthcare professional 50 may have access to a second programmer that is communicatively coupled (for example wirelessly coupled) to the first programmer. The patient's response or input on the first programmer may be sent to the second programmer to be viewable by the healthcare professional 50. The second programmer may also be configured to display information that is viewable by the healthcare professional 50 but not viewable by the patient 30, for example the patient evaluation score. Either or both the first and second programmer may be communicatively coupled to the monitor 70 as well.

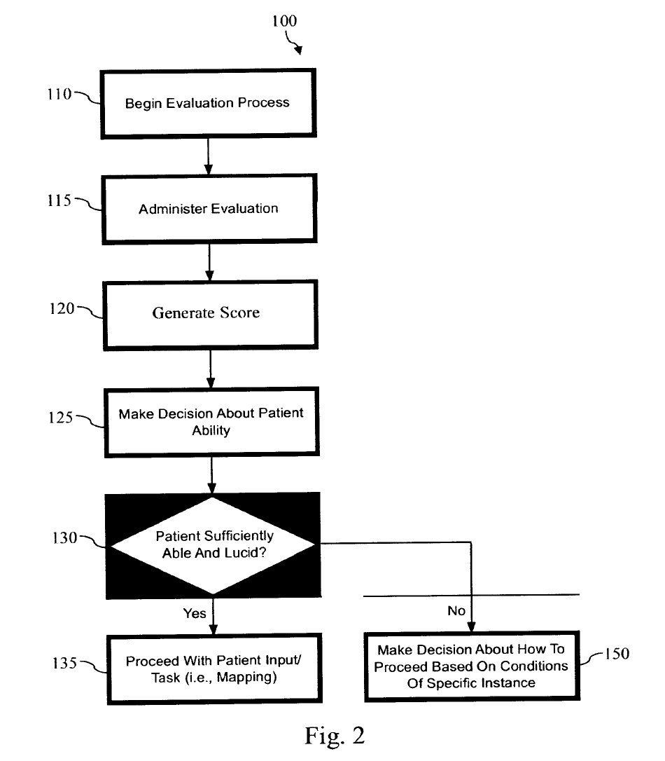

FIG. 2 is a flowchart illustrating a method 100 of performing the Cognition Evaluation process according to an embodiment of the present disclosure. The method 100 includes steps 110-150. At step 110, the Cognition Evaluation process begins. At step 115 of the method 100, the Cognition Evaluation is administered by a healthcare professional. At step 120, a patient score is generated based on the patient's response to the Cognition Evaluation. As discussed above, the patient score may be generated by the processing and analysis module. The score is received by the healthcare professional but may not be known to the patient. At step 125, the healthcare professional makes a decision about the patient's cognitive ability based on the score received from step 120. At step 130, a determination is made as to whether the patient is sufficiently able and lucid to provide reliable feedback to medical personnel.

If the answer from step 130 is yes, then the method 100 continues at step 135 to in which the patient performs suitable tasks so as to give input or feedback to a surgeon or healthcare professional. For example, the patient may be prompted to draw a pain map and/or a stimulation map. These maps may allow the surgeon to reposition the implanted medical device to optimize its therapeutic effects.

If the answer from step 130 is no, the method 100 proceeds to step 150, in which a decision is made about how to proceed further based on conditions of the specific instance.

Of course, the steps 110-150 illustrated in FIG. 2 and discussed above are merely one example of performing the Cognition Evaluation process. In other embodiments, additional steps may be performed in place of, or before/during/after the steps 110-150. These steps are not discussed herein for reasons of simplicity.

According to the various aspects of the present disclosure, the Cognition Evaluation process discussed above may be performed by one or more modules that may each be implemented on a separate device. For example, one of these modules may include a communications module, which may be an electronic device (for example a computer tablet or a similar device) through which instructions can be displayed or verbally communicated to the user or to someone else, such as a medical professional. As another example, another one of these modules may include a user input detection module, which may include a touchscreen display that is configured to detect or receive input from the user. The input from the user may include the user's finger movements on the touchscreen. As another example, another one of these modules may include a processing or analysis module that is configured to analyze the user's input received by the user input module and make an evaluation of the user's abilities based on the user's performance.

Each of the modules may include software code and hardware for storing and executing the software code, for example computer memory (for storing the code) and computer processor (for executing the code). In various embodiments, these modules may be implemented on separate devices or may be implemented on a single device. Also, each of the modules may include multiple sub-modules. For example, the communications module may include a sub-module for communicating with the patient and another sub-module for communicating with the medical professional but not necessarily to the patient.

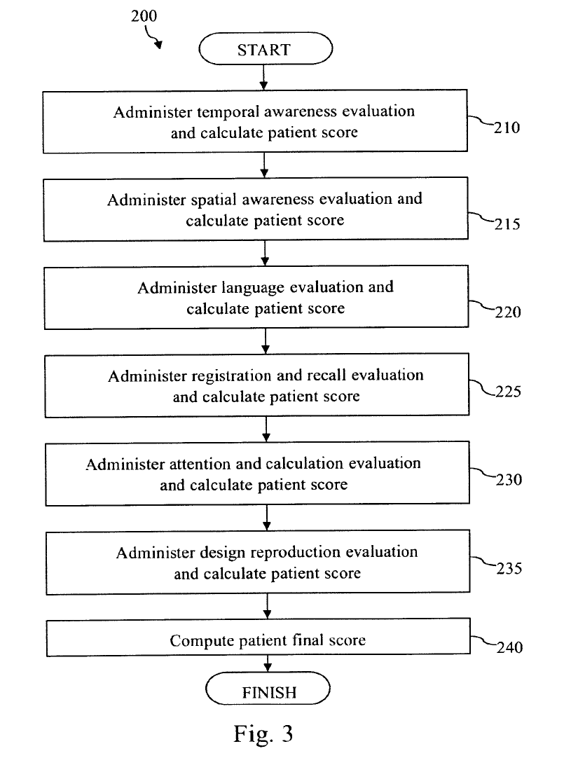

FIG. 3 is a flowchart illustrating a method 200 of performing a part of the Cognition Evaluation process according to an embodiment of the present disclosure. For example, in some embodiments, the method 200 may be used to administer a series of cognitive tests to the patient and to generate a score based on the patient's response. In other words, the method 200 may be used to implement steps 115-120 of the method 100 of FIG. 2.

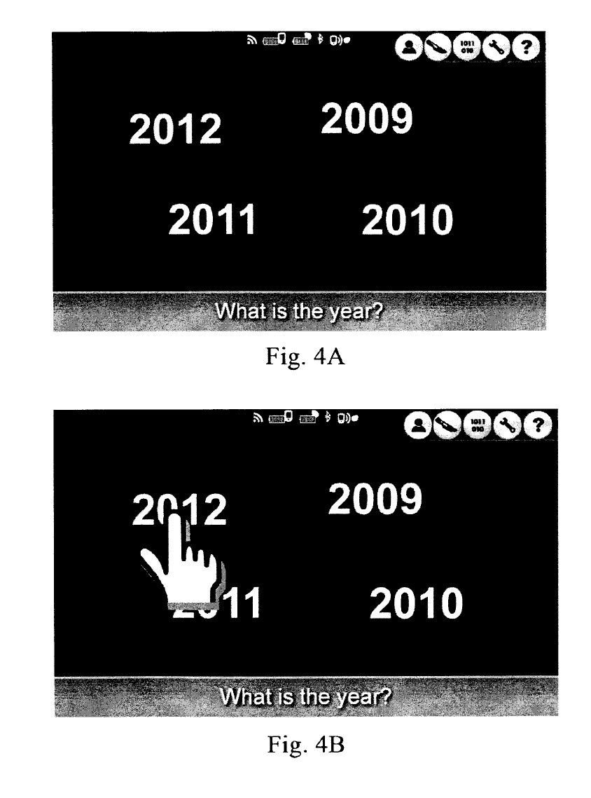

Referring to FIG. 3, the method 200 includes steps 210-240. Steps 210-235 each include a respective test or evaluation. It is understood that although FIG. 3 lists these steps 210-235 in an ordered sequential manner, these steps 210-235 may be performed in any sequence in actual implementation. Graphical examples of these evaluations are illustrated in FIGS. 4A-4B to 11A-11B. Therefore, the steps 210-235 of FIG. 3 will be discussed in view of FIGS. 4A-4B to 11A-11B below.

At step 210, a temporal awareness evaluation is administered. The temporal awareness evaluation is designed to determine whether the patient is able to place himself within a context of time. In other words, does the patient generally know when or what time it is? Temporal awareness is a part of the perception portion of the evaluation and is important for recognizing the progression of pain and stimulation over time. In some embodiments, the temporal awareness evaluation is used to assess the patient's ability to identify the current day of the week, date, month, year, and/or season.

For example, FIGS. 4A-4B illustrate example temporal awareness evaluation screenshots. In the temporal awareness evaluation discussed below, it is assumed that the patient has sufficient cognition with respect to numbers and the meanings thereof. In FIG. 4A, the patient is prompted with the question “what is the year?” while 2009, 2010, 2011, and 2012 are displayed on the screen. In FIG. 4B, the patient correctly responds by selecting the year 2012. It is understood that although FIGS. 4A-4B show the question in a text format and at the bottom of the screen, the question (or a series of questions) may be in a verbal form (e.g., a verbal question from the healthcare professional) and may be displayed elsewhere on the screen as well. The patient may also be allowed to respond either by touching the appropriate answer on a touchscreen device (for example on a clinician or patient programmer or another device capable of simulating the clinician or patient programmer), or by verbally communicating the answer, or via any other suitable means of communication.

Based on the detected response from the patient, a score is generated. If the response from the patient is correct, then a maximum score of X1 may be generated. If the response is incorrect, then a score less than X1 may be generated. In some embodiments, an incorrect response from the patient yields a zero score. In other embodiments, different scores less than X1 may be generated based on how “close” the patient's response is to the correct response. For example, if the correct answer is the year 2012, then the patient may get a higher score if he chose the year 2011 than if he chose 2009, since the year 2011 is closer to the year 2012 than the year 2009.

Referring back to FIG. 3, at step 215, a spatial awareness evaluation is administered. The spatial awareness evaluation is designed to determine whether the patient is able to ascertain his current location. In other words, does the patient generally know where he is? Spatial awareness is another part of the perception portion of the evaluation, as it tests whether the patient is able to recognize positions and locations. In some embodiments, the spatial awareness evaluation is used to assess the patient's ability to identify his current building, town, city, and/or country. In some embodiments, both spatial awareness and orientation awareness are used.

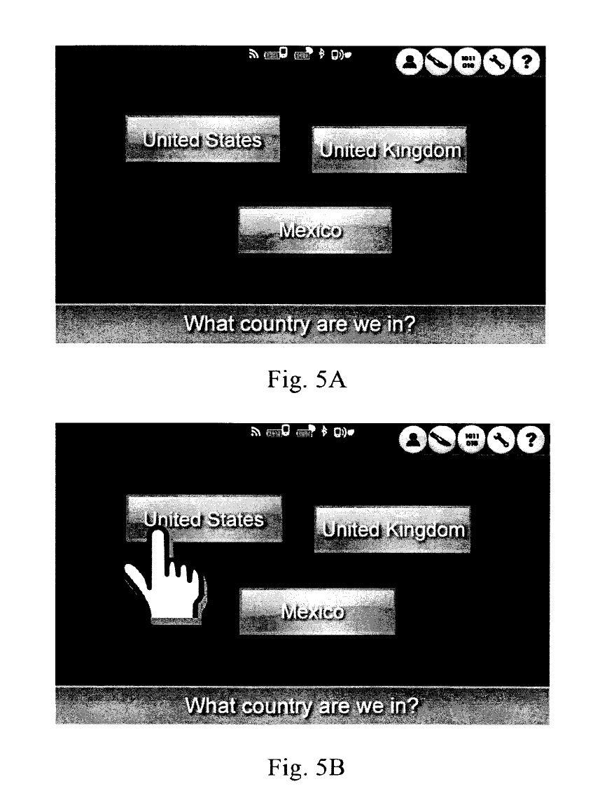

FIGS. 5A-5B illustrate example spatial awareness evaluation screenshots. In FIG. 5A, the patient is prompted with the question “what country are we in?” while countries such as United States, United Kingdom, and Mexico are displayed on the screen. Once again, the question is not limited to text but may be in any suitable format, and the patient may be allowed to respond via touchscreen or with a verbal answer, or with any other suitable means of communication. In FIG. 5B, the patient responds correctly by selecting the United States.

Based on the detected response from the patient, a score is generated. If the response from the patient is correct, then a maximum score of X2 may be generated. If the response is incorrect, then a score less than X2 may be generated. In some embodiments, an incorrect response from the patient yields a zero score. In other embodiments, different scores less than X2 may be generated based on how “close” the patient's response is to the correct response. For example, if the correct answer is the country United States, then the patient may get a higher score if he chose Mexico than if he chose United Kingdom, since Mexico is closer to the United States than the United Kingdom.

Referring back to FIG. 3, at step 220, a language evaluation is administered. The language evaluation is designed to determine whether the patient is able to follow basic written or verbal instructions. In other words, is the patient literate? The language may be English or any other suitable language. Language ability is yet another part of the perception portion of the evaluation, as it tests whether the patient is able to perceive information in the same way that the information is being conveyed. Language ability may be vital to a majority of interactions between a patient and a healthcare provider. In some embodiments, the language evaluation may include tests in which the patient is prompted to perform simple manipulations to common and/or well-known objects.

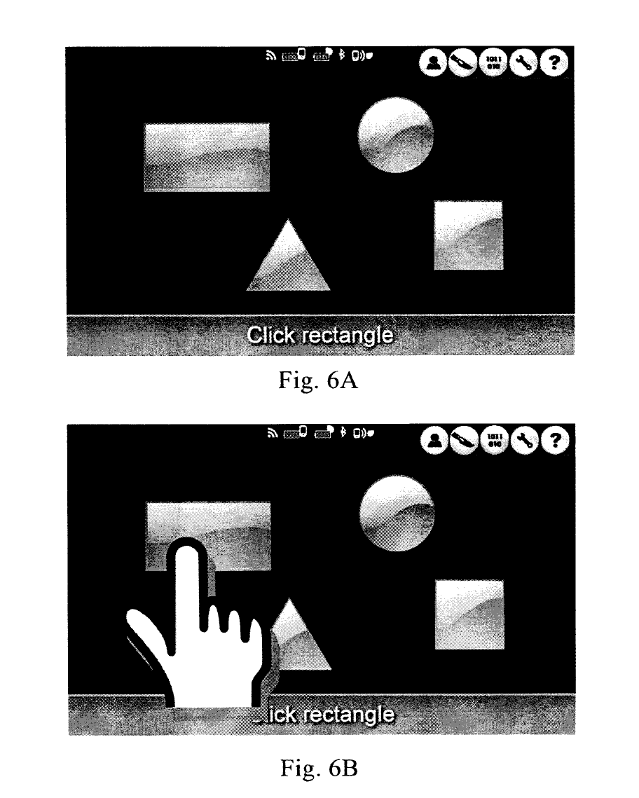

FIGS. 6A-6B illustrate example language evaluation screenshots. In FIG. 6A, the patient is prompted with the question “click rectangle” while shapes such as rectangle, circle, triangle, and square are displayed on the screen. Once again, the question is not limited to text but may be in any suitable format, and the patient may be allowed to respond via touchscreen or with a verbal answer, or with any other suitable means of communication. In FIG. 6B, the patient responds correctly by selecting the rectangle. In situations where the patient does not understand the meaning of “rectangle”, other more well-known shapes may be generated until the patient finds one that he understands.

Based on the detected response from the patient, a score is generated. If the response from the patient is correct, then a maximum score of X3 may be generated. If the response is incorrect, then a score less than X3 may be generated. In some embodiments, an incorrect response from the patient yields a zero score. In other embodiments, different scores less than X3 may be generated based on how “close” the patient's response is to the correct response. For example, if the correct answer is the rectangle, then the patient may get a higher score if he chose the square than if he chose the circle, since the square is closer to the rectangle than the circle.

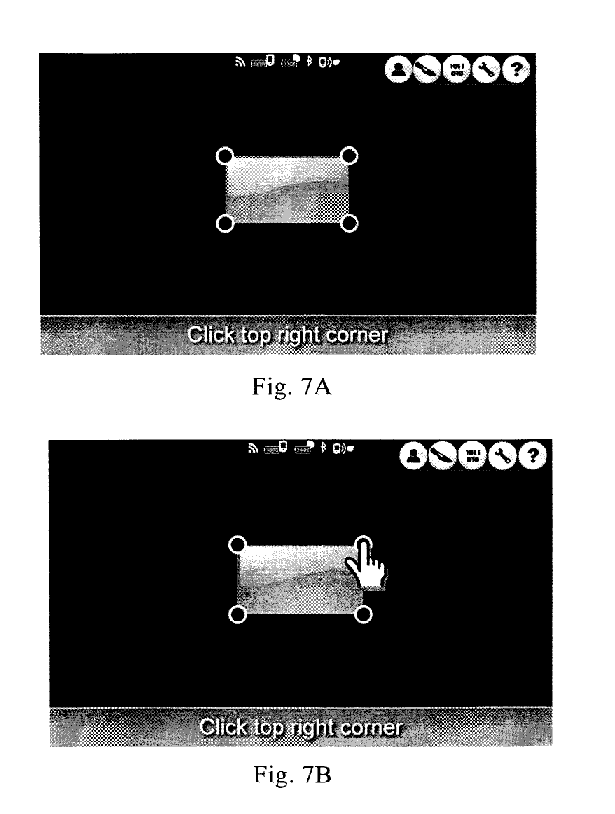

FIGS. 7A-7B illustrate other example language evaluation screenshots. In FIG. 7A, the patient is prompted with the question “click top right corner” while a rectangle with four highlighted corners is displayed on the screen. Once again, the question is not limited to text but may be in any suitable format, and the patient may be allowed to respond via touchscreen or with a verbal answer, or with any other suitable means of communication. In FIG. 7B, the patient responds correctly by selecting the top right corner.

Based on the detected response from the patient, a score is generated. If the response from the patient is correct, then a maximum score of X4 may be generated. If the response is incorrect, then a score less than X4 may be generated. In some embodiments, an incorrect response from the patient yields a zero score. In other embodiments, different scores less than X4 may be generated based on how “close” the patient's response is to the correct response. For example, since the correct answer is the top right corner, then the patient may get a higher score if he chose the bottom right corner than if he chose the bottom left corner, since the bottom right corner is closer to the top right corner than the bottom left corner.

Referring back to FIG. 3, at step 225, a registration evaluation is administered. The registration evaluation is designed to determine whether the patient has sufficient memory capacity. In other words, how well can the patient remember things? The patient may be asked to identify one or more unique images, then to remember the images later. This is part of the reasoning portion of the evaluation Memory lucidity is important for a patient in treatment in order to communicate with the healthcare provider whether pain (and/or perceived stimulation) is different, and in what way, from before. Memory lucidity is also important because the patient may be expected to recall menu items in a programmer in some scenarios.

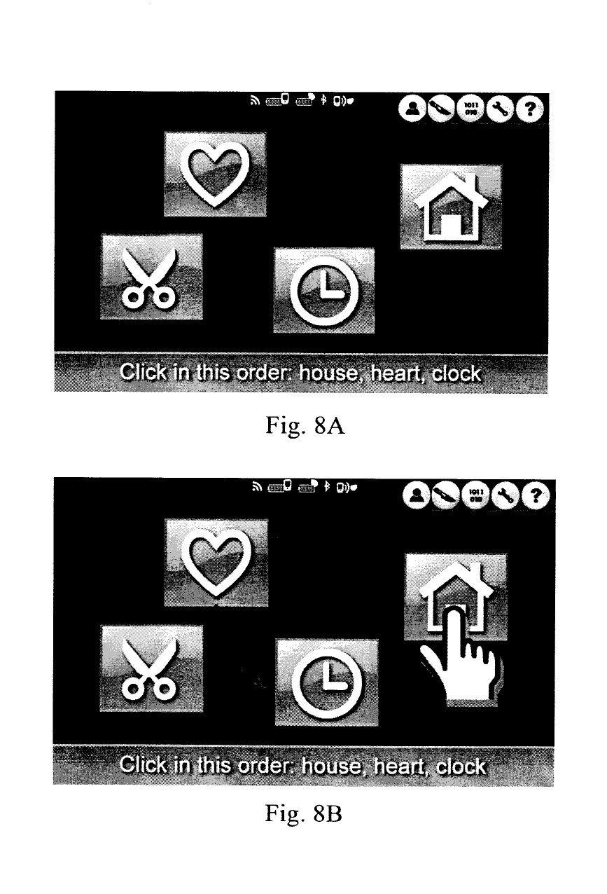

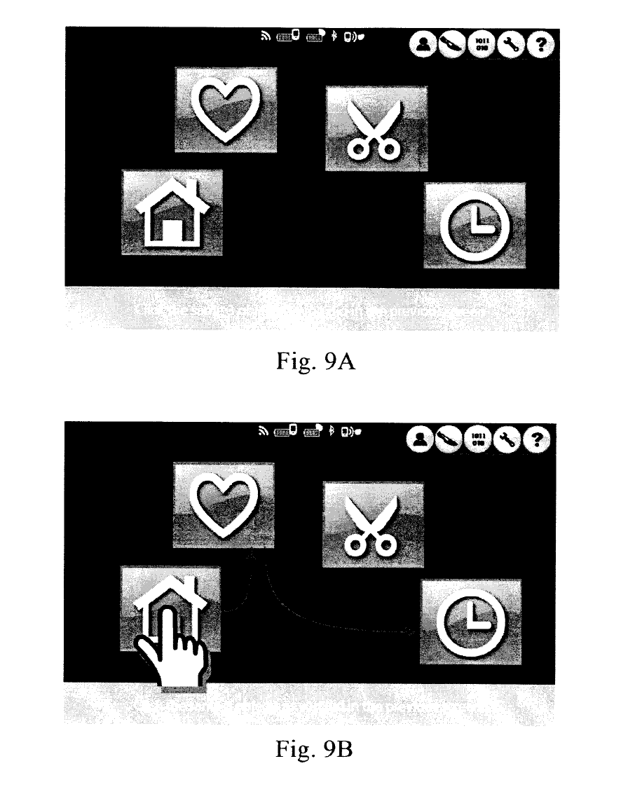

FIGS. 8A-8B and 9A-9B illustrate example registration and recall evaluation screenshots. In FIG. 8A, the patient is prompted with the question “click in this order: house, heart, clock” while objects representing scissors, heart, clock, and house are displayed on the screen. Once again, the question is not limited to text but may be in any suitable format, and the patient may be allowed to respond via touchscreen or with a verbal answer, or with any other suitable means of communication. In FIG. 8B, the patient responds correctly by clicking the house, heart, and clock, in that order.

At some time later, for example a few minutes later, or even after some of the other steps of the evaluation method have been performed, example screenshots of FIGS. 9A-9B are shown to the patient. In FIG. 9A, the patient is prompted with the question “click the same objects as your did in the previous screen” while the same objects representing scissors, heart, clock, and house are again displayed on the screen. In FIG. 9B, the patient responds correctly by clicking the house, heart, and clock, in that same specific order as he did before.

Based on the detected response from the patient, a score is generated. If the response from the patient is correct, then a maximum score of X5 may be generated. If the response is incorrect, then a score less than X5 may be generated. In some embodiments, an incorrect response from the patient yields a zero score. In other embodiments, different scores less than X5 may be generated based on how “close” the patient's response is to the correct response. For example, if the correct answer is clicking the house, heart, and clock in that specific order, then the patient may get a higher score if he clicked on the house, heart, and clock but not in that specific order, than if he clicked on totally incorrect objects and in a completely random order.

Referring back to FIG. 3, at step 230, an attention calculation evaluation is administered. The attention calculation evaluation is designed to determine whether the patient is able to think analytically. In other words, can the patient reason? The attention calculation evaluation is another part of the reasoning portion of the evaluation. Analytical ability is important for the patient to understand consequences of disparate courses of action, among other cause-and-effect situations and various other instances. In some embodiments, the attention calculation evaluation may include tests in which a patient is asked to count in reverse order from a pseudo random number within a certain range.

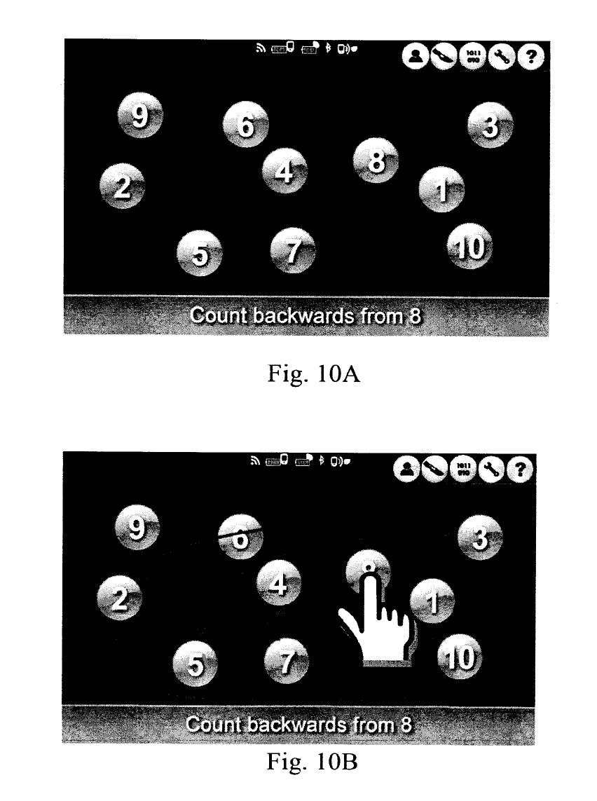

FIGS. 10A-10B illustrate example attention and calculation evaluation screenshots. In FIG. 10A, the patient is prompted with the question “count backwards from 8” while numbers from 1-10 are displayed on the screen. Once again, the question is not limited to text but may be in any suitable format, and the patient may be allowed to respond via touchscreen or with a verbal answer, or with any other suitable means of communication. In FIG. 10B, the patient responds correctly by clicking the numbers in reverse order from 8 to 1.

Based on the detected response from the patient, a score is generated. If the response from the patient is correct, then a maximum score of X6 may be generated. If the response is incorrect, then a score less than X6 may be generated. In some embodiments, an incorrect response from the patient yields a zero score. In other embodiments, different scores less than X6 may be generated based on how “close” the patient's response is to the correct response. For example, if the correct answer is clicking the numbers backwards from 8 to 1, then the patient may get a higher score if he clicked the numbers backwards from 8 to 2 than if he clicked on totally different numbers in a random sequence.

Referring back to FIG. 3, at step 235, an evaluation is administered to determine the patient's ability to understand and perform complex commands. In the embodiment illustrated herein, the complex command is a design reproduction task. Thus, the evaluation herein may be referred to as a design reproduction evaluation thereafter for the sake of providing an example. The design reproduction evaluation is configured to determine whether the patient has adequate fine motor skills and perception of shapes for certain tasks, such as producing a pain map and/or a stimulation map. Being able to accurately draw or produce a pain map and/or a stimulation map is important for the patient because that can partially determine the success of the surgical implant of the medical device such as the neurostimulator. For example, if the initial placement of the neurostimulator is not delivering sufficient stimulation to the tissue area where the patient is feeling pain, the patient can communicate what he is experiencing with the surgeon by drawing a pain map and/or a stimulation map. The surgeon may then adjust the placement of the neurostimulator based on the patient's feedback. This would not be possible if the patient lacks sufficient fine motor skills and perception of shapes to actually draw the pain map and/or the stimulation map. In some embodiments, the design reproduction evaluation may include tests in which a patient is shown one or more certain images and then asked to re-draw these images.

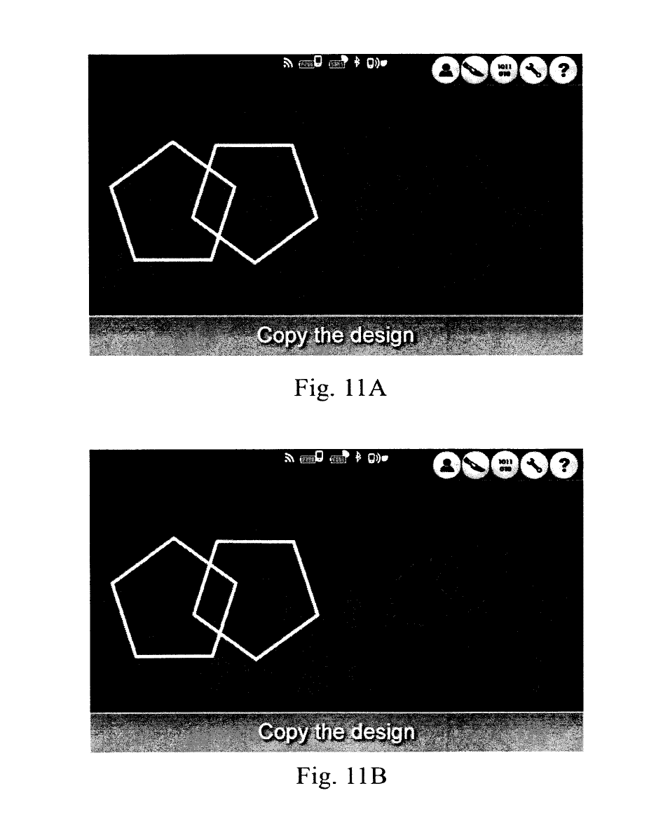

FIGS. 11A-11B illustrate example design reproduction evaluation screenshots. In FIG. 11A, the patient is prompted with the question “copy the design” while two pentagons are displayed in a certain arrangement on the left side of the screen. Once again, the question is not limited to text but may be in any suitable format, and the patient may be allowed to respond via touchscreen or with a verbal answer, or with any other suitable means of communication. In FIG. 11B, the patient responds correctly by drawing the two pentagons in the same approximate arrangement on the right side of the screen.

Based on the detected response from the patient, a score is generated. Depending on the degree of resemblance between the patient's drawing and the image that was given, a score up to X7 may be generated. For example, if the patient substantially draws two pentagons in substantially the same arrangement as the image on the left, then the maximum score of X7 may be generated. However, if the drawing produced by the patient does not sufficiently resemble pentagons or if they are not in the same approximate relative arrangement as shown in the left side of the screen, then a score less than X7 is generated. If the patient's drawing deviates too far from the image on the left side of the screen, a zero score may be generated.

Referring back to FIG. 3, after all the steps 210-235 of the Cognition Evaluation method have been performed, a final score X is computed for the patient in step 240. The final score X is a composite score that is a function of all the scores generated in each of the steps 210-235. In some embodiments, all the scores from steps 210-235 are added up, the sum of which is the final patient score X. In other embodiments, one or more scores from the steps 210-235 may be weighted more than others. For example, depending on the particular circumstances, it may be deemed that the patient's ability to remember and recall events is more important than the other aspects of the Cognition Evaluation method. Thus, the score X5 from the “registration and recall evaluation” step 225 may be applied a weighting coefficient greater than 1, for example 2 or 3. Thus, the final patient score X will have a greater contribution from the “registration and recall evaluation” score X5. Similarly, if one or more of the steps 210-235 is deemed to be less important, then a weighting coefficient less than 1 may be applied to their corresponding scores as well.

In any case, according to steps 120-130 of method 100 in FIG. 2, after the patient's final score X is computed, it is compared to a predetermined threshold score Y. If the score X is less than Y, that indicates the patient has failed the Cognition Evaluation, which means the patient is not sufficiently able and lucid. The process flow then proceeds to step 140, as discussed above with reference to FIG. 2. On the other hand, if the score X is greater than or equal to Y, that indicates the patient has passed the Cognition Evaluation, which means the patient is sufficiently able and lucid. Of course, it is understood that the threshold score Y and the comparison discussed above is merely one of the many embodiments. In other embodiments, no threshold score is used, and the patient's final score X may be subjectively used by the physician according to the knowledge of the patient. For example, if the patient does not speak English well (not his native language), then the physician may take that into account when evaluating the patient's abilities in view of the final score X. The process flow then proceeds to step 135, as discussed above with reference to FIG. 2.

It is understood that the series of cognitive evaluations 210-235 are merely examples. In other embodiments, different standardized evaluation techniques may be employed to design and implement each of the evaluations above, including but not limited to, temporal awareness, spatial awareness, language proficiency, memory capacity, attention and calculation ability, and design and reproduction ability.

### II. Usability Aptitude Evaluation

The Usability Aptitude Evaluation is used to assess a patient's ability to use a specific method of providing input. Stated differently, the Usability Aptitude Evaluation tests the patient in a manner consistent with the method and technique of providing patient input or feedback to a medical personnel. The paragraphs below describe a suggested test for a specific input technology, for example a model manipulation on a clinician programmer touchscreen, with the possible objective of creating pain and/or stimulation maps. However, the Usability Aptitude Evaluation is not limited to this test or technology. Rather, the Usability Aptitude Evaluation concept is applicable to any method, technology, or means of communication.

According to some embodiments, the clinician programmer touchscreen Usability Aptitude Evaluation includes a three-dimensional (3D) model manipulation and may be referred to as the “Model Usability Aptitude Evaluation” hereinafter. The Model Usability Aptitude Evaluation (and its equivalent score) may include three portions: spatial identification, graphical comprehension, and 3D model shading, as defined in Table 1 below.

TABLE 1
Model Usability Aptitude Evaluation Definitions

| | | Points |
|---|---|---|
| Component | Definition | Awarded |
| Spatial | Evaluates the user's ability to understand | 10 |
| Identification | and manipulate basic 2D or 3D shapes. | |
| Graphical | Evaluates the user's ability to read a | 10 |
| Comprehension | graph or infographic. | |
| 3D Model | Evaluates the user's ability to shade | 10 |
| Shading | a 3D model. | |

In some embodiments, the Usability Aptitude Evaluation may be administered in an environment similar to the one illustrated in FIG. 1 and discussed above. In other words, the Usability Aptitude Evaluation may be administered before, during, or immediately after surgery. In other embodiments, the Usability Aptitude Evaluation may be administered in any other suitable environment, for example in any situation where the healthcare professional is concerned about the patient's ability to sufficiently use a programmer that configures the implanted medical device.

In some embodiments, the Usability Aptitude Evaluation involves assigning the patient (or any other user) a standard set of tasks, and the patient's response to the tasks will be scored. Scores and an assessment about patient ability will then be presented to the healthcare professional. A patient needs to achieve a passing score in order to be deemed as capable of using the programmer. In some embodiments, the patient is not given access to the scores and the assessment. Similar to the Cognition Evaluation, the Usability Aptitude Evaluation may be designed to be game-like so as to better capture the attention of the patient.

To carry out the Usability Aptitude Evaluation process, a method similar to the method 100 of FIG. 2 may be performed. In other words, the Usability Aptitude Evaluation (which includes one or more tests) is administered to the patient, a composite score is generated based on the patient's response, and a determination is made (for example by the physician) as to whether the patient is sufficiently lucid and able to provide dependable medical feedback. If so, the patient may be asked to provide suitable input relevant to the surgery and placement of the implanted medical device. The input is provided using an electronic device such as the patient programmer or clinician programmer. If the patient fails the Usability Aptitude Evaluation and is not deemed lucid, the healthcare professional may then make decisions based on the specific conditions and circumstances.

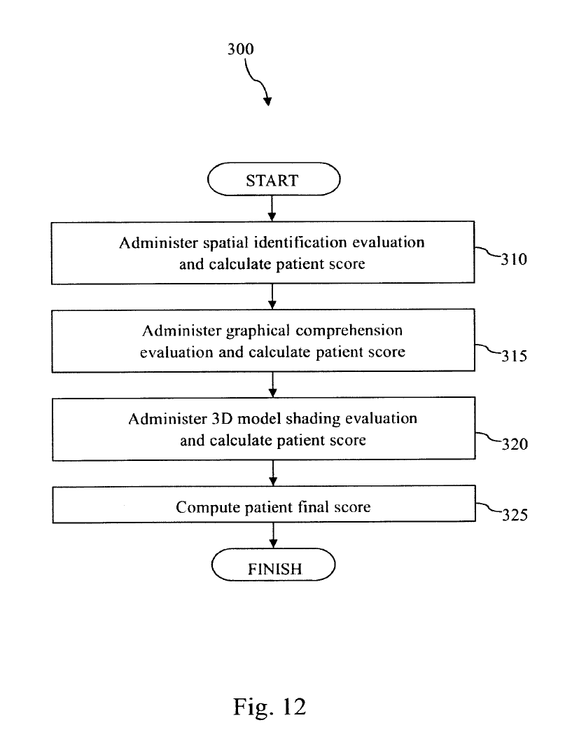

FIG. 12 is a flowchart illustrating a method 300 of performing a part of the Usability Aptitude Evaluation process according to an embodiment of the present disclosure. For example, in some embodiments, the method 300 may be used to administer a series of usability aptitude tests to the patient and to generate a score based on the patient's response.

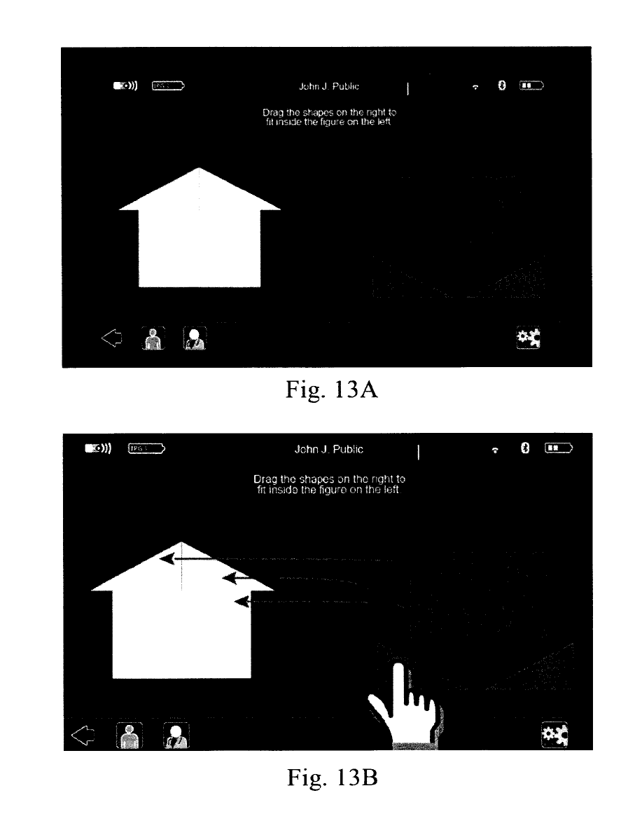

Referring to FIG. 12, the method 300 includes steps 310-325. Steps 310-320 each include a respective evaluation. It is understood that although FIG. 12 lists these steps 310-320 in an ordered sequential manner, these steps 310-320 may be performed in any sequence in actual implementation. Graphical examples of these evaluations are illustrated in FIGS. 13-15. Therefore, the steps 310-320 of FIG. 12 will be discussed in view of FIGS. 13-15 below.

At step 310, a spatial identification evaluation is administered. The spatial identification evaluation is designed to determine the patient's ability to understand and manipulate basic two-dimensional (2D) or three-dimensional (3D) shapes, or any other basic shapes. Spatial identification is employed in many patient input tasks, such as mapping, selecting, and dragging.

For example, FIGS. 13A-13B illustrate example spatial identification evaluation screenshots. In FIG. 13A, the patient is prompted with the instructions “drag the shapes on the right to fit inside the figure on the left.” The figure on the left is composed of two triangles on top of a rectangle. The shapes on the right include two triangles and a rectangle, but they are not properly laid out.

In FIG. 13B, the patient successfully completes the task by dragging (indicated by the arrows) each shape on the right to its correct counterpart on the left. It is understood that although FIGS. 13A-13B display the instructions in a text format, the instructions may be in a verbal form (e.g., a verbal instruction from the healthcare professional, or through a speaker from the clinician programmer) in other embodiments.

Based on the detected response from the patient, a score is generated. If the patient has successfully completed the task, then a maximum score of M1 may be generated. If the patient was unable to complete the task, then a score less than M1 may be generated. In some embodiments, an unsuccessful completion of the task yields a zero score. In other embodiments, different scores less than M1 may be generated based on how well the patient was able to complete the task. For example, even if the patient was not able to perfectly align each shape on the right to its counterpart on the left, he/she may receive a partial score less than M1 if he/she was able to align the shapes close enough.

Referring back to FIG. 12, at step 315, a graphical comprehension evaluation is administered. The graphical comprehension evaluation is designed to determine the patient's ability to read a graph or infographic, which may be a pie chart, as an example. In some embodiments, the graphical comprehension evaluation requires a patient's understanding of various types of charts and graphs, for example pie charts that indicate when two variables are balanced.

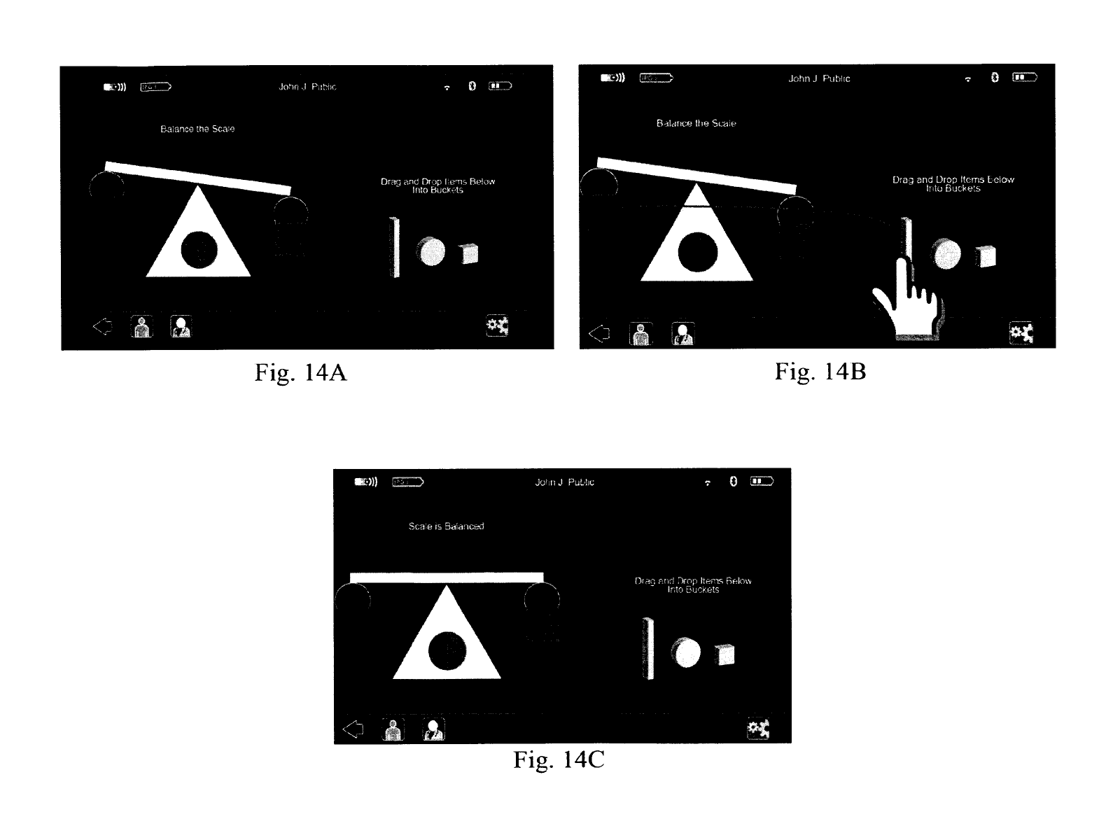

For example, FIGS. 14A-14B illustrate example graphical comprehension evaluation screenshots. In more detail, the patient is asked to balance a scale using different objects of varying perceived weights. Referring to FIG. 14A, the patient is shown an unbalanced scale on the left of the screen and a plurality of different objects having varying perceived weights on the right of the screen. For example, the perceived weight of each object may be a function of its size. The patient is prompted with the instructions “balance the scale” above the scale on the left of the screen and the instructions “drag and drop items below into buckets” above the objects on the right of the screen. Although not specifically shown herein for the sake of simplicity, it is understood that when an object is placed in the bucket, it may still be shown inside the bucket.

In FIG. 14B, the patient engages the task by selecting and dragging (indicated by the arrow) various objects on the right to the buckets the left. In FIG. 14C, the patient has successfully completed the task, and the scale is now balanced. Once again, although FIGS. 14A-14B display the instructions in a text format, the instructions may be in a verbal form (e.g., a verbal instruction from the healthcare professional) in other embodiments.

Based on the detected response from the patient, a score is generated. If the patient has successfully completed the task, then a maximum score of M2 may be generated. If the patient was unable to complete the task, then a score less than M2 may be generated. In some embodiments, an unsuccessful completion of the task yields a zero score. In other embodiments, different scores less than M2 may be generated based on how well the patient was able to complete the task. For example, even if the patient was not able to perfectly balance the scale, he may receive a partial score less than M2 if he was able to achieve a close enough balancing of the scale, or at least improve the balancing of the scale compared to its initial unbalanced state.

Referring back to FIG. 12, at step 320, a 3D model shading evaluation is administered. The 3D model shading evaluation is designed to assess the patient's ability to shade or orient a 3D model. These skills are used in patient input tasks, especially in the creation of pain maps and/or stimulation maps where the user has to orient the model and paint the request portions of the model. Thus, the patient should be capable of understanding how to interpret 3D models.

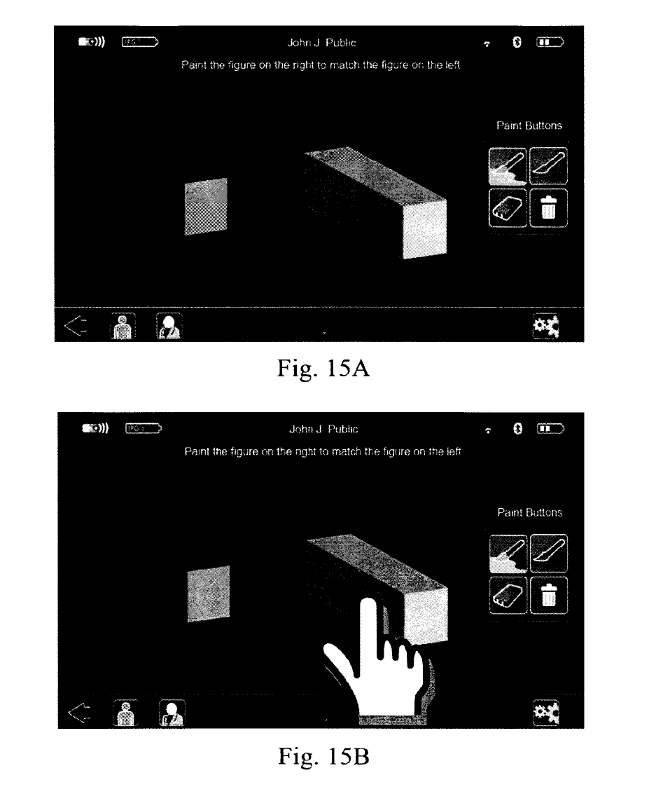

FIGS. 15A-15B illustrate example 3D model shading evaluation screenshots. Referring to FIG. 15A, the patient is shown a 3D polygon on the left of the screen, where different sides or surfaces of the polygon may be shaded with different colors. On the right of the screen, an unshaded 3D polygon that is similar in size and shape to the polygon on the left is displayed. Various paint buttons are also displayed. Each button has an easy-to-understanding function. For example, one button may indicate that it can be used to apply a first color to an object, another button may indicate that it can be used to apply a second color to an object, a third button may indicate that it can be used as an eraser, and another button may indicate that it can be used to delete objects or clear painting or shading. The patient is also prompted with the instructions “paint the figure on the right to match the figure on the left.”

In FIG. 15B, the patient engages the task by shading the polygon on the right side of the screen with appropriate colors. Once again, although FIGS. 15A-15B display the instructions in a text format, the instructions may be in a verbal form (e.g., a verbal instruction from the healthcare professional) in other embodiments.

Based on the detected response from the patient, a score is generated. If the patient has successfully completed the task, then a maximum score of M3 may be generated. If the patient was unable to complete the task, then a score less than M3 may be generated. In some embodiments, an unsuccessful completion of the task yields a zero score. In other embodiments, different scores less than M3 may be generated based on how well the patient was able to complete the task. For example, even if the patient was not able to perfectly shade the polygons according to the model on the left, he/she may receive a partial score less than M3 if he/she was able to achieve a partially correct shading of the polygon mesh (or other two or three dimensional visual objects) on the right.

It can be seen that the 3D model shading evaluation measures the patient's ability to alter an attribute or characteristic of a graphical model or object on the screen. In the example illustrated in FIGS. 15A-15B, the attribute being altered is the color (shading) of the 3D polygon. In some embodiments, other suitable evaluations may be made in which the patient is prompted to alter other attributes of a graphical model on the screen. For example, the patient may be prompted to resize, reshape, or rotate one or more 2D or 3D graphical models. These manipulations are not specifically illustrated herein for reasons of simplicity.

Referring back to FIG. 12, after all the steps 310-320 of the Usability Aptitude Evaluation have been performed, a final score M is computed for the patient in step 325. The final score M is a composite score that is a function of all the scores generated in each of the steps 310-320. In some embodiments, all the scores from steps 310-320 are added up, the sum of which is the final patient score M. In other embodiments, one or more scores from the steps 310-320 may be weighted more than others. For example, depending on the particular circumstances, it may be deemed that the patient's ability to shade 3D models is more important than the other aspects of the Usability Aptitude Evaluation method. Thus, the score M3 from the “3D model shading” step 320 may be applied a weighting coefficient greater than 1, for example 2 or 3. Thus, the final patient score M will have a greater contribution from the “3D model shading” score M3. Similarly, if one or more of the steps 310-320 is deemed to be less important, then a weighting coefficient less than 1 may be applied to their corresponding scores as well.

In any case, after the patient's final score M is computed, it is compared to a predetermined threshold score N. If the score M is less than N, that indicates the patient or user has failed the Usability Aptitude Evaluation, which means the patient is not sufficiently able to perform the required tasks involved in providing reliable medical feedback. For example, the patient is unlikely to draw an accurate pain map or a stimulation map representing the pain or stimulation he is experiencing. The healthcare professional may then decide to wait or make other appropriate decisions based on the circumstances.

On the other hand, if the score M is greater than or equal to N, that indicates the patient has passed the Usability Aptitude Evaluation, which means the patient is sufficiently able to perform the required tasks involved in providing reliable medical feedback. The patient may then be asked to carry out one or more of these tasks on an electronic device such as a clinician programmer or a patient programmer, and the results of which will be fed back to the healthcare professional and the surgeon. For example, the patient may be asked to draw a pain map or a stimulation map on a device containing a touchscreen (such as a clinician or patient programmer, or a computer tablet). This pain map or stimulation map may be shown to the healthcare professional administering the evaluations, or the surgeon who is performing the surgical procedure that implants a medical device inside the patient's body. Based on the pain map or stimulation map, the surgeon may adjust the placement or other suitable settings of the implanted medical device to optimize its therapeutic effects for the patient.

It is also understood that the Cognition Evaluation and the Usability Aptitude Evaluation may be performed at a time when the patient is completely sober and alert, for example before surgery. This may be done to establish a “baseline” of the patient's mental and physical capabilities. Thereafter, when the Cognition Evaluation and Usability Aptitude Evaluation are performed again during or after surgery, the healthcare professional may more accurately assess how much, if any, of the patient's mental and physical capabilities have been impaired, and thus how reliable the patient's feedback can be. In some embodiments, the analysis of the comparison between the patient's “baseline” evaluation results and the “impaired” results can be built in to the evaluation program.

It is also understood that although the tests are described above (and shown in FIGS. 4-11 and 13-15) in association with either the Cognition Evaluation or the Usability Aptitude Evaluation in the present embodiments, in certain other embodiments some of these tests normally associated with the Cognition Evaluation may be performed for the Usability Aptitude Evaluation, and vice versa. Furthermore, the Cognition Evaluation or the Usability Aptitude Evaluation may each include additional tests not illustrated herein for reasons of simplicity.

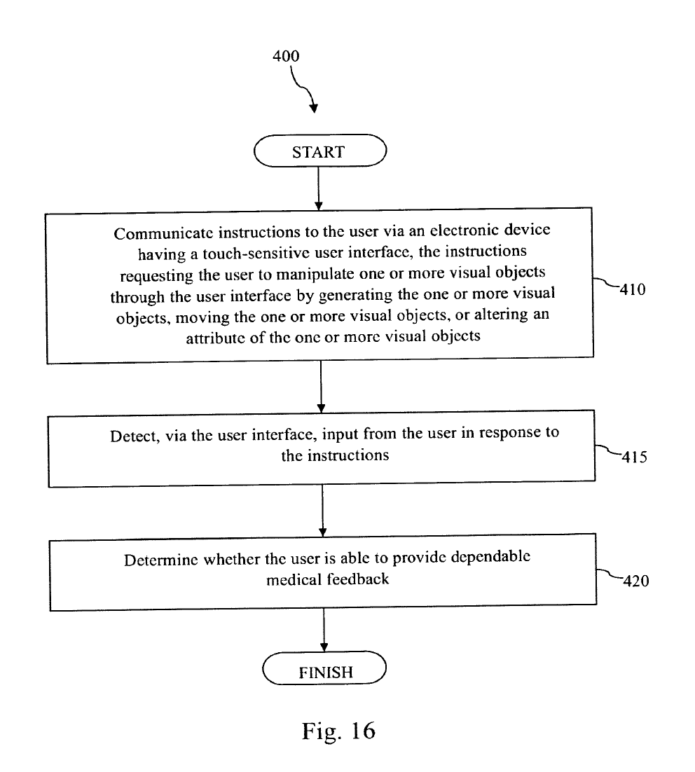

FIG. 16 is a flowchart illustrating a method 400 of interacting with a user. The method 400 includes a step 410, in which instructions are communicated to the user via an electronic device having a touch-sensitive user interface. The instructions request the user to manipulate one or more visual objects through the user interface by generating the one or more visual objects, moving the one or more visual objects, or altering an attribute of the one or more visual objects. In some embodiments, the generating the one or more visual objects includes representing the one or more objects according to a specified shape. In some embodiments, the moving the one or more visual objects includes dragging the one or more visual objects to a specified location. In some embodiments, the altering the attribute of the one or more visual objects includes resizing, reshaping, or coloring the one or more visual objects.

In some embodiments, the instructions are configured to measure one or more of the following cognitive and tactile capabilities of the user: temporal awareness, spatial awareness, language proficiency, memory capacity, attention and calculation ability, design and reproduction ability, spatial identification ability, graphical comprehension ability, and three-dimensional model shading ability. In some embodiments, the touch-sensitive user interface is displayed on a touch-sensitive screen of the electronic device.

The method 400 includes a step 415, in which input from the user in response to the instructions of step 410 is detected via the user interface. In some embodiments, the electronic device is a portable handheld device configured for wireless communications. In some embodiments, the electronic device includes one of: a clinician programmer, a patient programmer, and a computer tablet. In some embodiments, the user is a patient for whom a medical device is being, or has been, implanted inside the patient's body.

The method 400 includes a step 420 in which it is determined whether the user is able to provide dependable medical (or other types of) feedback. In some embodiments, the determination in step 420 is made by: generating a composite score based on the user's input to the instructions, and determining that the user is able to provide dependable medical feedback if the composite score is greater than or equal to a predefined threshold. In some embodiments, the determination in step 420 is made by determining whether the user is able to draw a reliable pain map or a stimulation map using the touch-sensitive screen.

It is understood that additional steps may be performed before, during, or after the steps 410-420 of the method 400. For example, in some embodiments, after the step 420 is performed, the result of the determination in step 420 is communicated to a medical professional. The communication to the medical professional may include electronically sending the result to a monitor external to the electronic device that is viewable by the medical professional but not by the user or patient.

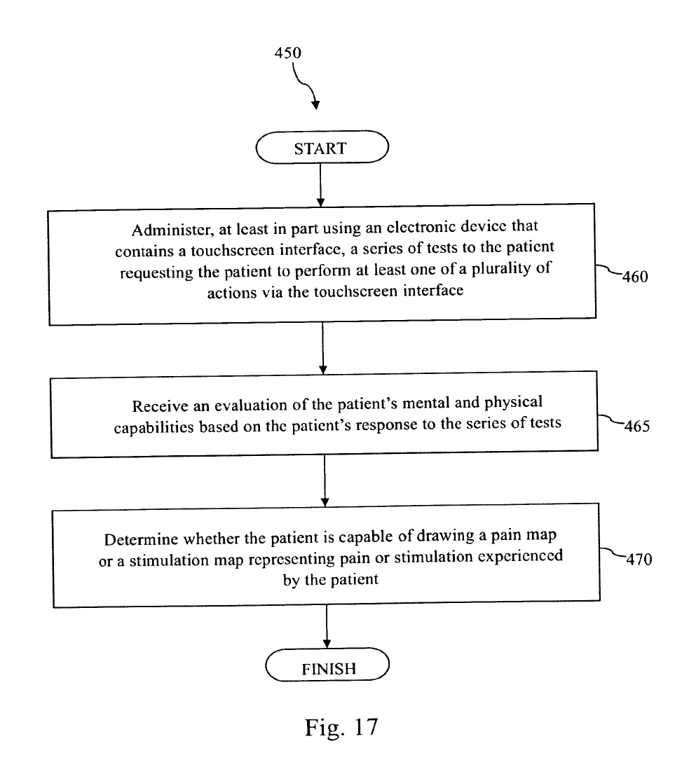

FIG. 17 is a flowchart of a method 450 of evaluating a patient's mental and physical capabilities. The method 450 includes a step 460 in which a series of tests is administered to the patient. The series of tests is administered at least in part using an electronic device that contains a touchscreen interface. The series of tests requests the patient to perform at least one of the following actions via the touchscreen interface: producing an image having a specified geometry; moving an image from a first location to a specified second location; resizing or reshaping an image; shading an image with a specified color; and clicking on a set of images according to a specified sequence. In some embodiments, the image includes a three-dimensional graphical model.

In some embodiments, at least one of the actions performed by the patient indicates one or more of the following cognitive and tactile capabilities of the patient: temporal awareness, spatial awareness, language proficiency, memory capacity, attention and calculation ability, design and reproduction ability, spatial identification ability, graphical comprehension ability, and three-dimensional model shading ability.

The method 450 includes a step 465 in which an evaluation of the patient's mental and physical capabilities is received based on the patient's response to the series of tests.

The method 450 includes a step 470 in which a determination is made as to whether the patient is capable of drawing a pain map or a stimulation map representing pain or stimulation experienced by the patient.

It is understood that additional steps may be performed before, during, or after the steps 460-470 of the method 450. For example, after it has been determined that the patient is capable of drawing a pain map or a stimulation map as a result of the step 470, another step is performed in which the patient is asked to draw the pain map or the stimulation map. An instrument is provided to the patient to draw the pain map or the stimulation map. In some embodiments, the instrument has an interface that is the same as, or substantially similar to, the touchscreen interface of the electronic device. In some embodiments, the instrument is a clinician programmer, a patient programmer, or an electronic tablet.

In some embodiments, the steps 460, 465, and 470 are performed during or after a surgical procedure in which a surgeon implants a medical device inside the patient's body. The surgeon may be asked to adjust the medical device implanted inside the patient's body based on the pain map or stimulation map drawn by the patient. In some embodiments, the steps 460, 465, and 470 are performed in a non-sterile part of an operating room in which the surgical procedure is performed.

### III. Implanted Medical Device System

As discussed above, some example contexts in which the Cognition Evaluation and the Usability Aptitude Evaluation may take place involves the implantation of a medical device. For example, during or after the surgery in which the surgeon implants a medical device such as a neurostimulator in the patient's body, the patient may undergo the Cognition Evaluation and Usability Aptitude Evaluation. These evaluations are done so that the healthcare professional may evaluate whether the patient has sufficient mental and physical functions to be able to understand and manipulate a programming device used to configure the implanted medical device. As such, the patient may offer the surgeon reliable feedback (e.g., meaningful answers to questions and accurate drawings of pain/stimulation maps) regarding the implanted medical device, for example by drawing pain and/or stimulation maps. That way, the surgeon may adjust the implanted medical device according to the patient's feedback. Also by taking and passing these evaluations, the patient may prove he is able to use the programming device safely, and may therefore be given permission to use it to configure the implanted medical device after surgery.

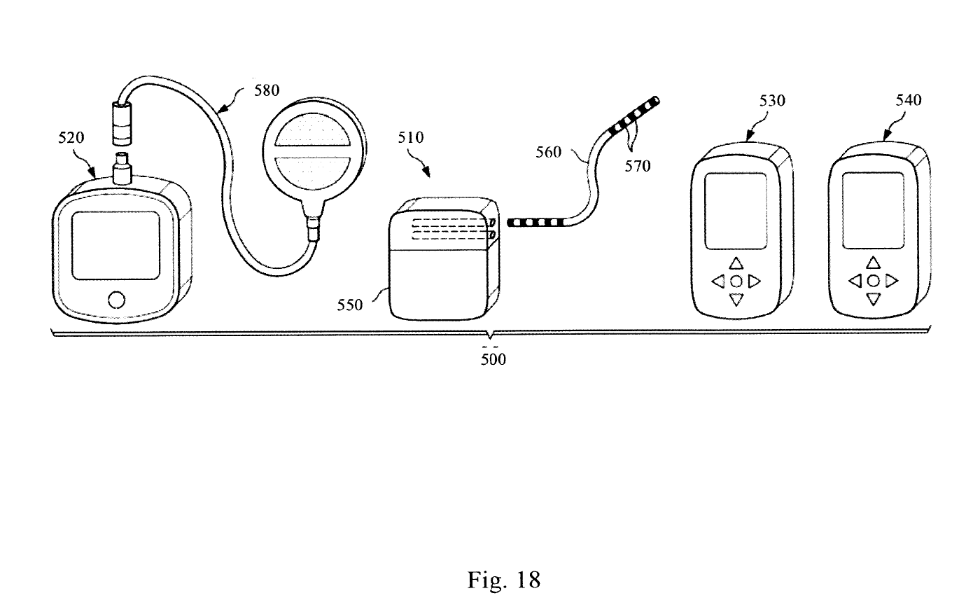

FIG. 18 illustrates a simplified block diagram of an implanted medical device system 500 that pertains to the discussions above. The implanted medical system 500 includes an implantable medical device 510, an external charger 520, a patient programmer 530, and a clinician programmer 540. The drawings for these devices are simplified, for example the patient programmer 530 and the clinician programmer 540 may not look substantially identical in real life. The implantable medical device 510 is the device that is implanted in a patient's body tissue, as discussed above. In the illustrated embodiment, the implantable medical device 510 includes an implanted pulse generator (IPG) 550 (a type of neurostimulator) that is coupled to one end of an implanted lead 560. The other end of the implanted lead 560 includes multiple electrode surfaces 570 through which electrical current is applied to a desired part of a body tissue. The implanted lead 560 incorporates electrical conductors to provide a path for that current to travel to the body tissue from the IPG 550. Although only one implanted lead 560 is shown in FIG. 18, it is understood that a plurality of implanted leads may be attached to the IPG 550.

The external charger 520 of the medical device system 500 provides electrical power to the IPG 550. The electrical power may be delivered through a charging coil 580. The IPG 550 may also incorporate power-storage components such as a battery or capacitor so that it may be powered independently of the external charger 520 for a period of time, for example from a day to a month, depending on the power requirements of the therapeutic electrical stimulation delivered by the IPG.

The patient programmer 530 and the clinician programmer 540 may be portable handheld devices that can be used to configure the IPG 550 so that the IPG 550 can operate in a certain way. The patient programmer 530 is used by the patient in whom the IPG 550 is implanted. The patient may adjust the parameters of the stimulation, such as by selecting a program, changing its amplitude, frequency, and other parameters, and by turning stimulation on and off. The clinician programmer 540 of the medical device system 500 is used by a medical personnel to configure the other system components and to adjust stimulation parameters that the patient is not permitted to control, such as by setting up stimulation programs among which the patient may choose, selecting the active set of electrode surfaces in a given program, and by setting upper and lower limits for the patient's adjustments of amplitude, frequency, and other parameters.

According to the present disclosure, the Cognition Evaluation and the Usability Aptitude Evaluation discussed above with reference to FIGS. 1-17 may be implemented in an electronic device such as the patient programmer 530 or the clinician programmer 540 of FIG. 18. For example, the Cognition Evaluation and the Usability Aptitude Evaluation may be implemented on the patient programmer and/or the clinician programmer through a software interface.

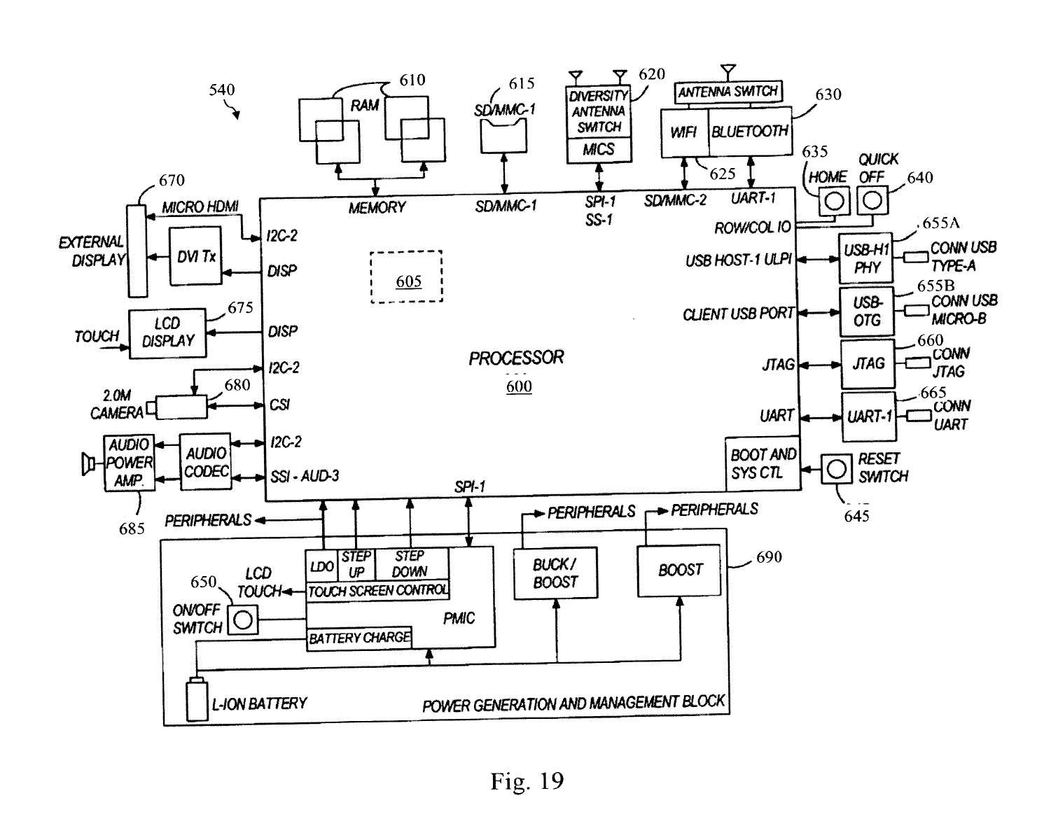

FIG. 19 shows a block diagram of one embodiment of the clinician programmer (CP) 540 (FIG. 18) that can be used to perform the Cognition Evaluation the Usability Aptitude Evaluation. It is understood, however, that other embodiments of the CP may be used to perform these evaluations too.

The CP includes a printed circuit board (“PCB”) that is populated with a plurality of electrical and electronic components that provide power, operational control, and protection to the CP. With reference to FIG. 19, the CP includes a processor 600. The processor 600 controls the CP. In one construction, the processor 600 is an applications processor model i.MX515 available from Freescale Semiconductor®. More specifically, the i.MX515 applications processor has internal instruction and data caches, multimedia capabilities, external memory interfacing, and interfacing flexibility. Further information regarding the i.MX515 applications processor can be found in, for example, the “IMX51CEC, Rev. 4” data sheet dated August 2010 and published by Freescale Semiconductor® at www.freescale.com. The content of the data sheet is incorporated herein by reference. Of course, other processing units, such as other microprocessors, microcontrollers, digital signal processors, etc., can be used in place of the processor 600.

The CP includes memory, which can be internal to the processor 600 (e.g., memory 605), external to the processor 600 (e.g., memory 610), or a combination of both. Exemplary memory include a read-only memory (“ROM”), a random access memory (“RAM”), an electrically erasable programmable read-only memory (“EEPROM”), a flash memory, a hard disk, or another suitable magnetic, optical, physical, or electronic memory device. The processor 600 executes software that is capable of being stored in the RAM (e.g., during execution), the ROM (e.g., on a generally permanent basis), or another non-transitory computer readable medium such as another memory or a disc. The CP also includes input/output (“I/O”) systems that include routines for transferring information between components within the processor 600 and other components of the CP or external to the CP.

Software included in the implementation of the CP is stored in the memory 605 of the processor 600, RAM 610, ROM 615, or external to the CP. The software includes, for example, firmware, one or more applications, program data, one or more program modules, and other executable instructions. The processor 600 is configured to retrieve from memory and execute, among other things, instructions related to the control processes and methods described below for the CP.

One memory shown in FIG. 19 is memory 610, which may be a double data rate (DDR2) synchronous dynamic random access memory (SDRAM) for storing data relating to and captured during the operation of the CP. In addition, a secure digital (SD) multimedia card (MMC) may be coupled to the CP for transferring data from the CP to the memory card via slot 615. Of course, other types of data storage devices may be used in place of the data storage devices shown in FIG. 19.

The CP includes multiple bi-directional radio communication capabilities. Specific wireless portions included with the CP are a Medical Implant Communication Service (MICS) bi-directional radio communication portion 620, a WiFi bi-directional radio communication portion 625, and a Bluetooth bi-directional radio communication portion 630. The MICS portion 620 includes a MICS communication interface, an antenna switch, and a related antenna, all of which allows wireless communication using the MICS specification. The WiFi portion 625 and Bluetooth portion 630 include a WiFi communication interface, a Bluetooth communication interface, an antenna switch, and a related antenna all of which allows wireless communication following the WiFi Alliance standard and Bluetooth Special Interest Group standard. Of course, other wireless local area network (WLAN) standards and wireless personal area networks (WPAN) standards can be used with the CP.

The CP includes three hard buttons: a “home” button 635 for returning the CP to a home screen for the device, a “quick off” button 640 for quickly deactivating stimulation IPG, and a “reset” button 645 for rebooting the CP. The CP also includes an “ON/OFF” switch 650, which is part of the power generation and management block (discussed below).

The CP includes multiple communication portions for wired communication. Exemplary circuitry and ports for receiving a wired connector include a portion and related port for supporting universal serial bus (USB) connectivity 655, including a Type A port and a Micro-B port; a portion and related port for supporting Joint Test Action Group (JTAG) connectivity 660, and a portion and related port for supporting universal asynchronous receiver/transmitter (UART) connectivity 665. Of course, other wired communication standards and connectivity can be used with or in place of the types shown in FIG. 19.

Another device connectable to the CP, and therefore supported by the CP, is an external display. The connection to the external display can be made via a micro High-Definition Multimedia Interface (HDMI) 670, which provides a compact audio/video interface for transmitting uncompressed digital data to the external display. The use of the HDMI connection 670 allows the CP to transmit video (and audio) communication to an external display. This may be beneficial in situations where others (e.g., the surgeon) may want to view the information being viewed by the healthcare professional. The surgeon typically has no visual access to the CP in the operating room unless an external screen is provided. The HDMI connection 670 allows the surgeon to view information from the CP, thereby allowing greater communication between the clinician and the surgeon. For a specific example, the HDMI connection 670 can broadcast a high definition television signal that allows the surgeon to view the same information that is shown on the LCD (discussed below) of the CP.

The CP includes a touch screen I/O device 675 for providing a user interface with the clinician. The touch screen display 675 can be a liquid crystal display (LCD) having a resistive, capacitive, or similar touch-screen technology. It is envisioned that multitouch capabilities can be used with the touch screen display 675 depending on the type of technology used.

The CP includes a camera 680 allowing the device to take pictures or video. The resulting image files can be used to document a procedure or an aspect of the procedure. Other devices can be coupled to the CP to provide further information, such as scanners or RFID detection. Similarly, the CP includes an audio portion 685 having an audio codec circuit, audio power amplifier, and related speaker for providing audio communication to the user, such as the clinician or the surgeon.

The CP further includes a power generation and management block 690. The power block 690 has a power source (e.g., a lithium-ion battery) and a power supply for providing multiple power voltages to the processor, LCD touch screen, and peripherals.

In one embodiment, the CP is a handheld computing tablet with touch screen capabilities. The tablet is a portable personal computer with a touch screen, which is typically the primary input device. However, an external keyboard or mouse can be attached to the CP. The tablet allows for mobile functionality not associated with even typical laptop personal computers. The hardware may include a Graphical Processing Unit (GPU) in order to speed up the user experience. An Ethernet port (not shown in FIG. 19) may also be included for data transfer.

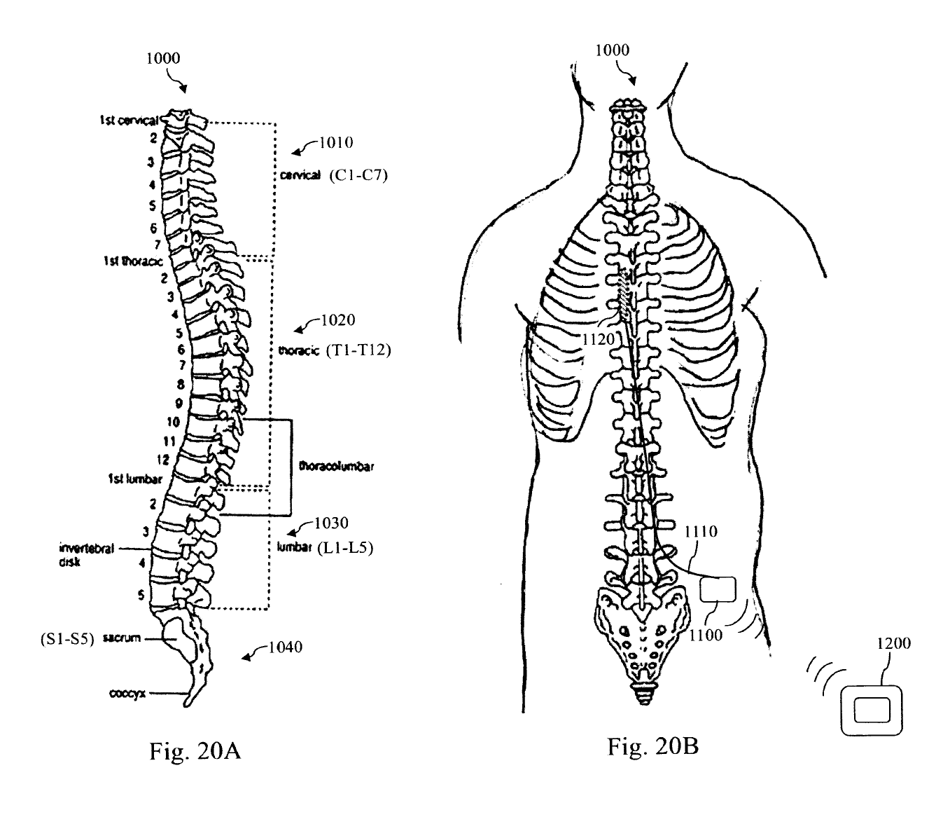

FIG. 20A is a side view of a spine 1000, and FIG. 20B is a posterior view of the spine 1000. The spine 1000 includes a cervical region 1010, a thoracic region 1020, a lumbar region 1030, and a sacrococcygeal region 1040. The cervical region 1010 includes the top 7 vertebrae, which may be designated with C1-C7. The thoracic region 1020 includes the next 12 vertebrae below the cervical region 1010, which may be designated with T1-T12. The lumbar region 1030 includes the final 5 “true” vertebrae, which may be designated with L1-L5. The sacrococcygeal region 1040 includes 9 fused vertebrae that make up the sacrum and the coccyx. The fused vertebrae of the sacrum may be designated with S1-S5.

Neural tissue (not illustrated for the sake of simplicity) branch off from the spinal cord through spaces between the vertebrae. The neural tissue can be individually and selectively stimulated in accordance with various aspects of the present disclosure. For example, referring to FIG. 20B, an IPG device 1100 is implanted inside the body. The IPG device 1100 may include a neurostimulator device. A conductive lead 1110 is electrically coupled to the circuitry inside the IPG device 1100. The conductive lead 1110 may be removably coupled to the IPG device 1100 through a connector, for example. A distal end of the conductive lead 1110 is attached to one or more electrodes 1120. The electrodes 1120 are implanted adjacent to a desired nerve tissue in the thoracic region 1020. Using well-established and known techniques in the art, the distal end of the lead 1110 with its accompanying electrodes may be positioned along or near the epidural space of the spinal cord. It is understood that although only one conductive lead 1110 is shown herein for the sake of simplicity, more than one conductive lead 1110 and corresponding electrodes 1120 may be implanted and connected to the IPG device 1100.

The electrodes 1120 deliver current drawn from the current sources in the IPG device 1100, therefore generating an electric field near the neural tissue. The electric field stimulates the neural tissue to accomplish its intended functions. For example, the neural stimulation may alleviate pain in an embodiment. In other embodiments, a stimulator may be placed in different locations throughout the body and may be programmed to address a variety of problems, including for example but without limitation; prevention or reduction of epileptic seizures, weight control or regulation of heart beats.

It is understood that the IPG device 1100, the lead 1110, and the electrodes 1120 may be implanted completely inside the body, may be positioned completely outside the body or may have only one or more components implanted within the body while other components remain outside the body. When they are implanted inside the body, the implant location may be adjusted (e.g., anywhere along the spine 1000) to deliver the intended therapeutic effects of spinal cord electrical stimulation in a desired region of the spine. Furthermore, it is understood that the IPG device 1100 may be controlled by a patient programmer or a clinician programmer 1200, the implementation of which may be similar to the clinician programmer shown in FIG. 19. It is also understood that the Cognition Evaluation and Usability Aptitude Evaluation processes may be performed to a patient during, or after, the implantation of the IPG 1100.

The foregoing has outlined features of several embodiments so that those skilled in the art may better understand the detailed description that follows. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. An electronic device, comprising:
a touchscreen display configured to receive input from a user;
a memory storage component configured to store programming code; and
a computer processor configured to execute the programming code to perform an evaluation of the user's mental and physical abilities, wherein the evaluation includes:
prompting the user to perform a plurality of tasks, wherein at least one of the tasks prompts the user to manipulate one or more graphical models shown on the touchscreen display according to predefined instructions;
detecting, via the touchscreen display, responses from the user for the tasks; and
determining, based on the detected responses, whether the user is mentally and physically fit to provide reliable feedback to medical personnel.

2. The electronic device of claim 1, wherein at least one of the tasks prompts the user to move, via the touchscreen display, the one or more graphical models to one or more appropriate locations.

3. The electronic device of claim 1, wherein at least one of the tasks prompts the user to perform color shading, via the touchscreen display, to a three-dimensional graphical model.

4. The electronic device of claim 1, wherein at least one of the tasks prompts the user to comprehend an infographic item shown on the touchscreen display.

5. The electronic device of claim 1, wherein at least one of the tasks is configured to evaluate a cognitive ability of the user.

6. The electronic device of claim 5, wherein the cognitive ability of the user includes at least one of: temporal awareness, spatial awareness, language proficiency, memory capacity, attention and calculation ability, and design and reproduction ability.

7. The electronic device of claim 1, wherein the electronic device is a portable handheld device.

8. The electronic device of claim 7, wherein the electronic device includes one of: a clinician programmer and a patient programmer that are each capable of configuring an implantable medical device.

9. The electronic device of claim 1, wherein the determining comprises:
generating a respective score for the detected responses for each task;
computing a composite score that is a function of all the respective scores;
comparing the composite score with a threshold score;
deciding that the user is fit to provide reliable feedback to medical personnel if the composite score meets or exceeds the threshold score; and
deciding that the user is unfit to provide reliable feedback to medical personnel if the composite score is less than the threshold score.

10. The electronic device of claim 1, wherein the touchscreen display is configured to be updated to provide a representation of a mental and physical fitness of the user in response to the evaluation.

11. A medical system, comprising:
a first communications module configured to instruct a patient to perform a series of tasks designed to evaluate the patient's cognitive and tactile functions;
an input detection module configured to detect the patient's performance of the tasks, including detecting clicking and dragging movements made by the patient on a touchscreen;
a processing and analysis module configured to analyze the patient's performance of the tasks and in response thereto produce an evaluation of the patient's cognitive and tactile functions; and
a second communications module configured to display a representation of the evaluation of the patient's cognitive and tactile functions to a medical professional.

12. The medical system of claim 11, wherein:
the first communications module, the input detection module, and the processing and analysis module are implemented collectively in a portable handheld electronic device; and
the second communications module includes a monitor external to the portable handheld electronic device.

13. The medical system of claim 12, wherein the portable handheld electronic device includes a clinician programmer or a patient programmer each configured to program an active medical device implantable in the patient's body.

14. The medical system of claim 13, wherein the clinician programmer or the patient programmer is configured to program with the active medical device through a wireless communication protocol.

15. The medical system of claim 11, wherein the series of tasks include tasks that evaluate one or more of the following cognitive and tactile functions of the patient: temporal awareness, spatial awareness, language proficiency, memory capacity, attention and calculation ability, design and reproduction ability, spatial identification ability, graphical comprehension ability, and three-dimensional model shading ability.

16. The medical system of claim 11, wherein the processing and analysis module is configured to analyze the patient's performance by assigning a sub-score to the patient's performance of each task.

17. The medical system of claim 16, wherein the processing and analysis module is configured to evaluate the patient's cognitive and tactile functions by comparing a cumulative score of the patient's performance for the series of tasks with a baseline criteria, the cumulative score being a function of all the sub-scores.

18. The medical system of claim 11, wherein the second communications module is configured to display the representation in a manner such that it is not viewable by the patient.

19. The medical system of claim 11, wherein at least one of the first communications module, the input detection module, the processing and analysis module, and the second communications module contains a computer processing unit configured to execute programming instructions.

20. A method of interacting with a user, comprising:
communicating instructions to the user via an electronic device having a touch-sensitive user interface, the instructions requesting the user to manipulate one or more visual objects through the user interface by generating the one or more visual objects, moving the one or more visual objects, or altering an attribute of the one or more visual objects;
detecting, via the user interface, input from the user in response to the instructions; and
determining whether the user is able to provide dependable medical feedback.

21. The method of claim 20, wherein:
the generating the one or more visual objects comprises representing the one or more objects according to a specified shape;
the moving the one or more visual objects comprises dragging the one or more visual objects to a specified location; and
the altering the attribute of the one or more visual objects comprises resizing, reshaping, or coloring the one or more visual objects.

22. The method of claim 20, wherein the instructions are configured to measure one or more of the following cognitive and tactile capabilities of the user: temporal awareness, spatial awareness, language proficiency, memory capacity, attention and calculation ability, design and reproduction ability, spatial identification ability, graphical comprehension ability, and three-dimensional model shading ability.

23. The method of claim 20, further comprising: communicating a result of the determining to a medical professional.

24. The method of claim 23, wherein the communicating the result comprises electronically sending the result to a monitor external to the electronic device that is viewable by the medical professional but not by the user.

25. The method of claim 20, wherein the determining comprises:
generating a composite score based on the user's input to the instructions; and
determining that the user is able to provide dependable medical feedback if the composite score is greater than or equal to a predefined threshold.

26. The method of claim 20, wherein the touch-sensitive user interface is displayed on a touch-sensitive screen of the electronic device.

27. The method of claim 20, wherein the determining comprises determining whether the user is able to draw a reliable pain map or a stimulation map using the touch-sensitive screen.

28. The method of claim 20, wherein the electronic device is a portable handheld device configured for wireless communications.

29. The method of claim 28, wherein the electronic device includes one of: a clinician programmer, a patient programmer, and a computer tablet.

30. The method of claim 20, wherein the user is a patient for whom a medical device is being, or has been, implanted inside the patient's body.

31. A method of evaluating a patient's mental and physical capabilities, comprising:
administering, at least in part using an electronic device that contains a touchscreen interface, a series of tests to the patient requesting the patient to perform at least one of the following actions via the touchscreen interface:
producing an image having a specified geometry;
moving an image from a first location to a specified second location;
resizing or reshaping an image;
shading an image with a specified color; and
clicking on a set of images according to a specified sequence;
receiving an evaluation of the patient's mental and physical capabilities based on the patient's response to the series of tests; and
determining whether the patient is capable of drawing a pain map or a stimulation map representing pain or stimulation experienced by the patient.

32. The method of claim 31, further comprising: asking the patient to draw the pain map or the stimulation map once it has been determined that the patient is capable of doing so.

33. The method of claim 32, wherein the asking the patient comprises providing an instrument for the patient to draw the pain map or the stimulation map, the instrument having an interface that is the same as, or substantially similar to, the touchscreen interface of the electronic device.

34. The method of claim 33, wherein the instrument is a clinician programmer, a patient programmer, or an electronic tablet.

35. The method of claim 31, wherein the administering, the receiving, and the determining are performed during or after a surgical procedure in which a surgeon implants a medical device inside the patient's body; and further comprising: asking the surgeon to adjust the medical device implanted inside the patient's body based on the pain map or stimulation map drawn by the patient.

36. The method of claim 35, wherein the wherein the administering, the receiving, and the determining are performed in a non-sterile part of an operating room in which the surgical procedure is performed.

37. The method of claim 31, wherein the image includes a three-dimensional graphical model.

38. The method of claim 31, wherein the at least one of the actions performed by the patient indicates one or more of the following cognitive and tactile capabilities of the patient: temporal awareness, spatial awareness, language proficiency, memory capacity, attention and calculation ability, design and reproduction ability, spatial identification ability, graphical comprehension ability, and three-dimensional model shading ability.
